(19) United States
(12) Patent Application Publication
(10) Pub. No.: US 2002/0169356 A1
(43) Pub. Date: Nov. 14, 2002

(54) Method of treating the symptoms of scleroderma

(76) Inventor: Jesse Ross, Great Neck, NY (US)

Correspondence Address: MYRON AMER, P.C., Suite 310, 114 Old Country Road, Mineola, NY, 11501, US

(21) Appl. No.: 09/853,540

(22) Filed: May 14, 2001

(51) Int. Cl. A61N 1/00

(52) U.S. Cl. 600/14

(57) ABSTRACT

Using to a first and second advantage for scleroderma a known treatment of a high frequency electromagnetic field, the first advantage being that the rest period between the pulses is approximately twenty-four times as great as the duration of each pulse, so that any heat that might be accumulated in the patient during the occurrence of the pulse has many times longer for its dissipation, thereby providing a treatment which is not harmful to the patient, and now a second advantage that it can be applied to an exact location of the patient, wherein the site of the scleroderma which can visually determined is correlated to the exact site of the applied treatment.

### BACKGROUND OF THE INVENTION

The present invention relates generally to a new treatment to relieve the symptoms of scleroderma, which will provide noteworthy quality of life benefits without deleterious consequences.

Scleroderma, a condition preventing a normal lifestyle, is a chronic hardening and thickening of tissue, which may be a finding in several different diseases, occurring in a localized area and frequently confined to the skin and subcutaneous tissue, although it can occur in organ tissue. The cause of scleroderma is unknown, and there is no cure; but treatments can ease symptoms.

These treatments include, for localized scleroderma, skin protection measures such as cold-water room humidifiers, superfatted soaps, and lubricants and moisturizers to help prevent dry skin. Topical corticosteroids may also help.

With systemic scleroderma, additional therapies may include aspirin or other nonsteroidal anti-inflammatory drugs to treat joint pain and swelling. Corticosteroids may be prescribed to treat muscle, lung or joint problems.

Physicians also frequently recommend exercises to help keep a patient's skin and joints flexible and maintain range of motion and better blood flow.

Improved blood flow in a scleroderma-affected localized area is a desired effective treatment, but heretofore difficult to administer because the increase in blood flow is attendant by an increase in blood volume, and the latter increases temperature of the tissue which is deleterious to the patient's condition.

### SUMMARY OF THE INVENTION

Broadly, it is an object of the present invention to overcome the foregoing and other shortcomings of the prior art.

More particularly, it is an object to use to advantage the localized site of scleroderma as a site of an externally administered treatment which increases blood flow without an attendant increase in blood volume, all as will be better understood as the description proceeds.

### BRIEF DESCRIPTION OF THE DRAWINGS

The description of the invention which follows, together with the accompanying drawings should not be construed as limiting the invention to the example shown and described, because those skilled in the art to which this invention appertains will be able to devise other forms thereof within the ambit of the appended claims.

FIG. 1 is a perspective view of an apparatus for generating an electromagnetic field for practicing the within inventive method;

FIG. 2 is an illustration of a microphotograph of blood prior to the subjection to high frequency oscillation; and

FIG. 3 is another microphotograph illustration of the blood of FIG. 2 but after subjection to the high frequency oscillation and showing a pearl chain formation of the nutritive blood elements.

### DESCRIPTION OF THE PREFERRED AND ILLUSTRATED EMBODIMENTS OF THE INVENTION

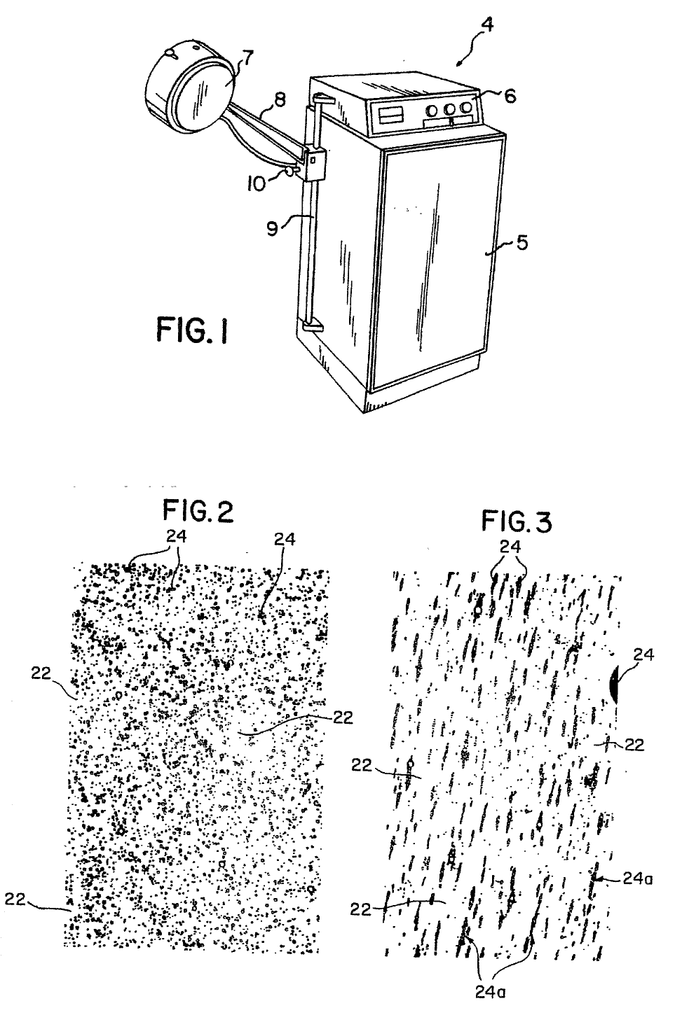

Shown in FIG. 1 is an athermapeutic apparatus for the generation of pulsed high frequency (short-wave) high peak power electromagnetic energy, to which a patient is subjected, of a type which is now well known to the art wherein the pulse frequency and duration is of such nature that the total time period during which electromagnetic energy can actually penetrate 6 to 8 inches into the body of a patient, and is so short that despite the comparatively high instantaneous energy level of the pulsed power it is unaccompanied by heat generation because the time for heat dissipation is many times longer than the heat accumulation. The athermapeutic apparatus 4 as therein shown comprises a cabinet 5 provided with a control panel 6 for regulating the pulse repetition rate and pulse duration, timer setting, etc., and having a treatment head 7. Such treatment head is carried by an arm 8 to which it is pivotally connected, and with the arm in turn being reciprocally and axially movable on a tubular support 9 and secured in any desired adjusted position relative to the support 9 by a locking screw 10.

Apparatus 4 will be understood to generate an electromagnetic field having a pulse duration and frequency which is fixed at sixty five microseconds and for pulse frequencies of from eighty to six hundred pulses per second, so that even at its maximum setting the total peak energy of nine hundred seventy-five watts maximum is of such short duration that the average power is only thirty-eight watts at maximum. Accordingly, at the maximum pulse rate of six hundred pulses per second the rest period between the pulses is approximately twenty-five times as great as the duration of each pulse, so that any heat that might be accompanied by the pulse in the patient during the occurrence of the pulse has many times longer for its dissipation by blood flow, thereby providing a treatment which is not harmful to the patient.

In the treatment use of the apparatus 4, the electromagnetic field utilized might typically have the following specific parameters:

1. A frequency of 27.12 megahertz (11 meter band);

2. A pulse repetition rate of 80 to 600 pulses per second;

3. A pulse width of 65 microseconds;

4. A power range, per pulse, of between 293 and 975 watts;

5. A duty cycle between 1/2 of 1% to 3.9%; and

6. A square pulse, with a rise and fall time less than 1%.

Underlying the present invention is the recognition that the generated electromagnetic field of apparatus 4 can be used to advantage to relieve the symptoms of scleroderma, an end use not heretofore known, without adverse side effects. The symptoms of scleroderma manifested typically as simple “dry skin,” or thickening and hardening of the skin, loss of elasticity, numbness, pain or color change, to mention but a few, but most are of the nature of being visually observed and localized. As a consequence, the scleroderma-affected area in most cases is readily visually determined and, in accordance with the present invention, readily determines the treatment site for the application of the electromagnetic field of apparatus 4. When internal organs evidence scleroderma, this treatment can penetrate to treat the internal site.

The head 7 of apparatus 4 is positioned so its electromagnetic field can penetrate in relation to the treatment site, the selection as explained being to make accessible to the generated electromagnetic field of the patient's circulatory system blood-flow to skin and subcutaneous tissue manifesting symptoms of scleroderma.

The effect of the penetration of this electromagnetic field on blood flow is best understood from FIGS. 2 and 3 to which reference should now be made. On FIGS. 2 and 3, the blank or unoccupied areas, individually and collectively designated 22 will be understood to be the fluid content of the blood and the occupied areas, also individually and collectively designated 24, will be understood to be the other components of which the blood is composed, such as lymph, chyle, plasma, and other body fluids.

By comparison of FIG. 2 before subjection to the electromagnetic field, to FIG. 3 after subjection, it should be readily observable that the pattern of FIG. 2 is a random dispersion of the blood fluid and nutritive elements contents 22, 24 and that in FIG. 3, the blood components 24 have assumed a chain formation, more particularly designed 24A, which formulation is known in the parlance of the art as the “Pearl-chain Formulation.”

A physical noteworthy attribute provided by the Pearl-chain formulation 24A is its longitudinal orientation which, during blood flow in the longitudinal direction is flow with minimum resistance which is manifested as an increase in blood flow or velocity, and will assume an agglomerated pattern (now shown) of fat globules favoring the increase in blood flow.

More particularly, using 10 as a base rate, this electromagnetic method increased blood velocity to 1.75 times the testing pulse. It was noted that no measurable increase of heat occurs during treatment and this is maintained from 1 to 8 hours. These reported favorable results were achieved using an athermapeutic apparatus commercially available from Diapulse Corporation of America, of Great Neck, N.Y. 11023-2420, said athermapeutic apparatus being described and illustrated in U.S. Pat. No. 3,043,310 for “Treatment Head For Athermapeutic Apparatus” issued on Jul. 10, 1962, which by this reference is incorporated herein in its entirety pursuant to MPEP 2163.07(b).

The increase in blood flow or velocity is without an attendant increase in volume, the latter being undesirable since it increases skin and tissue temperature and has a deleterious impact on the scleroderma condition.

While the apparatus for practicing with the inventive method, as well as said method herein shown and disclosed in detail is fully capable of attaining the objectives and providing the advantages hereinbefore stated, it is to be understood that it is merely illustrative of the presently preferred embodiment of the invention and that no limitations are intended to the detail of construction or design herein shown other than as defined in the appended claims.

What is claimed is:

1. A method of treating the symptoms of scleroderma comprising the steps of selecting as a site for treatment a determined localized manifestation of symptoms of scleroderma, generating an electromagnetic field into said selected site for treatment with operating parameters of a frequency of 27.12 megahertz, a pulse repetition rate of 80 to 600 pulses per second, a pulse width of 65 microseconds, a power range, per pulse of between 193 and 975 watts, a duty cycle between 1/1 of 1% to 3.9%, and a square pulse, with a rise and fall time less than 1%, affecting blood vessels in said selected site for treatment with said generated electromagnetic field, aligning all contents of said blood in a longitudinal orientation, and flowing in a longitudinal direction through said arteries, veins, capillaries and microcirculation, said electromagnetic field affecting blood flow, whereby blood flow in skin and subcutaneous tissue at said treatment site is increased to a symptom-relieving amount without increase in blood volume as might increase temperature of said skin and subcutaneous tissue and have a deleterious consequence on the scleroderma condition.
